(19) United States
(12) Patent Application Publication
(10) Pub. No.: US 2024/0342442 A1
(43) Pub. Date: Oct. 17, 2024

(54) DRESSING SYSTEM

(71) Applicant: Luke Blake, Terre Haute, IN (US)

(72) Inventor: Luke Blake, Terre Haute, IN (US)

(21) Appl. No.: 18/634,827

(22) Filed: Apr. 12, 2024

Related U.S. Application Data
(63) Continuation-in-part of application No. 17/210,445, filed on Mar. 23, 2021.
Application No. 17/210,445 is a continuation-in-part of application No. 16/175,912, filed on Oct. 31, 2018, now Pat. No. 10,953,199.
Application No. 16/175,912 is a continuation-in-part of application No. 15/802,528, filed on Nov. 3, 2017, now Pat. No. 10,874,471.
Application No. 15/802,528 is a continuation-in-part of application No. 15/377,146, filed on Dec. 13, 2016, now Pat. No. 10,117,717.
(60) Provisional application No. 63/553,191, filed on Feb. 14, 2024.
(60) Provisional application No. 63/533,301, filed on Aug. 17, 2023.
(60) Provisional application No. 63/458,685, filed on Apr. 12, 2023.

(51) Int. Cl. A61M 25/02; A61F 13/02

(52) U.S. Cl. CPC A61M 25/02; A61F 13/0259; A61M 2025/0273

(57) ABSTRACT

Dressing systems and methods of using the same are included herein. An exemplary dressing system can comprise a top layer comprising a first top layer element and a second top layer element, the first top layer element comprising a central area of a transparent window film having a first adhesive surrounded by the second top layer element comprising a border area of a polyurethane fabric having the first adhesive or a second adhesive, a bottom layer comprising a first bottom layer element and a second bottom layer element, the first bottom layer element comprising a second central area of the transparent window film having the first adhesive surrounded by the second bottom layer element comprising a border area of the polyurethane fabric having the first adhesive or the second adhesive, and a carrier configured to engage the top layer and the bottom layer.

### PRIORITY AND RELATED APPLICATIONS

The present application is related to, and claims the priority benefit of, a) U.S. provisional patent application Ser. No. 63/553,191, filed on Feb. 14, 2024, b) U.S. provisional patent application Ser. No. 63/533,301, filed on Aug. 17, 2023, and c) U.S. provisional patent application Ser. No. 63/458,685, filed on Apr. 12, 2023, and is related to, claims the priority benefit of, and is a continuation-in-part application of, U.S. patent application Ser. No. 17/210,445, filed Mar. 23, 2021, which is related to, claims the priority benefit of, and is a continuation-in-part patent application of, U.S. patent application Ser. No. 16/175,912, filed Oct. 31, 2018 and issued as U.S. Pat. No. 10,953,199 on Mar. 23, 2021, which is related to, claims the priority benefit of, and is a continuation-in-part application of, U.S. patent application Ser. No. 15/802,528, filed Nov. 3, 2017 and issued as U.S. Pat. No. 10,874,471 on Dec. 29, 2020, which is related to, claims the priority benefit of, and is a continuation-in-part application of, U.S. patent application Ser. No. 15/377,146, filed on Dec. 13, 2016 and issued as U.S. Pat. No. 10,177,717 on Nov. 6, 2018. The contents of each of the aforementioned patent applications and patents are hereby incorporated into the present disclosure in their entirety.

### BRIEF SUMMARY

The present disclosure includes disclosure of a dressing system, including bandage/dressing systems used to cover indwelling intravenous and pain block catheter entry points and general wounds. It is a two-layer bandage comprising a bottom layer and a top layer. The top layer may be removed and replaced with a replacement top layer when needed to protect the bottom layer and allow the bottom layer to protect of wound from contamination.

The present disclosure includes disclosure of a dressing system, comprising a top layer comprising a first top layer element and a second top layer element, the first top layer element comprising a central area of a transparent window film having a first adhesive surrounded by the second top layer element comprising a border area of a polyurethane fabric having the first adhesive or a second adhesive, a bottom layer comprising a first bottom layer element and a second bottom layer element, the first bottom layer element comprising a second central area of the transparent window film having the first adhesive surrounded by the second bottom layer element comprising a border area of the polyurethane fabric having the first adhesive or the second adhesive, and a carrier configured to engage the top layer and the bottom layer.

The present disclosure includes disclosure of a dressing system, wherein the bottom layer is sized and shaped to be smaller than the central area of the top layer element.

The present disclosure includes disclosure of a dressing system, wherein the bottom layer is completely visible within the central area of the top layer element.

The present disclosure includes disclosure of a dressing system, wherein the first adhesive comprises a low-tack adhesive applied to a lower surface of the first top layer element in a pattern.

The present disclosure includes disclosure of a dressing system, wherein the second adhesive comprises a pressure sensitive adhesive blended with an adhesive additive.

The present disclosure includes disclosure of a dressing system, wherein the carrier comprises a middle portion and two side portions.

The present disclosure includes disclosure of a dressing system, wherein the carrier comprises two pull tabs positioned thereon, each pull tab adjacent a middle portion and a side portion of the carrier.

The present disclosure includes disclosure of a dressing system, wherein the carrier comprises a peripheral piece and a central piece located within the peripheral piece.

The present disclosure includes disclosure of a dressing system, wherein the peripheral piece comprises one pull tab.

The present disclosure includes disclosure of a dressing system, wherein the peripheral piece comprises two handles.

The present disclosure includes disclosure of a dressing system, wherein the peripheral piece comprises two pull tabs.

The present disclosure includes disclosure of a dressing system, wherein the carrier comprises a one-piece carrier.

The present disclosure includes disclosure of a dressing system, wherein the one-piece carrier comprises a peripheral border and two pull tabs.

The present disclosure includes disclosure of a dressing system, further comprising a catheter securement system, comprising a window frame, a catheter securement, and a securement carrier.

The present disclosure includes disclosure of a dressing system, wherein the window frame and the catheter securement each have a cut out for a catheter.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows an in-use view of a dressing system whereby the top layer dressing is in the process of being removed from the bottom layer dressing, according to an exemplary embodiment of the present disclosure;

FIG. 2 shows an exploded top-down view of a dressing system, according to an exemplary embodiment of the present disclosure;

FIG. 3 shows an exploded bottom-up view of a dressing system, according to an exemplary embodiment of the present disclosure;

FIG. 4 shows an exploded top-down view of a dressing system, according to an exemplary embodiment of the present disclosure;

FIG. 5 shows an exploded bottom-up view of a dressing system, according to an exemplary embodiment of the present disclosure;

FIG. 6 shows a bottom view of a dressing system with central area removed from said dressing system, according to an exemplary embodiment of the present disclosure;

FIG. 7 shows a top view (upper portion of figure) and a bottom view (lower portion of figure) of a top layer dressing of the present disclosure, according to an exemplary embodiment of the present disclosure;

FIG. 8 shows a top view (upper portion of figure) and a bottom view (lower portion of figure) of a bottom layer dressing, according to an exemplary embodiment of the present disclosure;

FIG. 9 shows bottom views of a dressing system of the present disclosure with central area present (upper portion of figure) and removed (lower portion of figure), according to an exemplary embodiment of the present disclosure;

FIG. 10 shows a top view of a prior art dressing;

FIG. 11 shows a top view of a prior art dressing;

FIG. 12 shows an exploded top-down view of a dressing system, according to an exemplary embodiment of the present disclosure;

FIG. 13 shows an exploded bottom-up view of a dressing system, according to an exemplary embodiment of the present disclosure;

FIG. 14 shows an exploded top-down view of a catheter securement system, according to an exemplary embodiment of the present disclosure;

FIG. 15 shows an exploded bottom-up view of a catheter securement system, according to an exemplary embodiment of the present disclosure;

FIG. 16 shows an exploded top-down view of a dressing system, according to an exemplary embodiment of the present disclosure;

FIG. 17 shows an exploded bottom-up view of a dressing system, according to an exemplary embodiment of the present disclosure;

FIG. 18 shows an exploded top-down view of a replacement top layer dressing of a dressing system, according to an exemplary embodiment of the present disclosure;

FIG. 19 shows an exploded bottom-up view of a replacement top layer dressing of a dressing system, according to an exemplary embodiment of the present disclosure;

FIG. 20 shows an exploded top-down view of a catheter securement system, according to an exemplary embodiment of the present disclosure;

FIG. 21 shows an exploded bottom-up view of a catheter securement system, according to an exemplary embodiment of the present disclosure;

FIG. 22 shows a top-down view of a dressing system being used to cover an insertion site of an indwelling catheter into the skin, according to an exemplary embodiment of the present disclosure;

FIG. 23 shows an exploded top-down view of a dressing system, according to an exemplary embodiment of the present disclosure;

FIG. 24 shows an exploded bottom-up view of a dressing system, according to an exemplary embodiment of the present disclosure;

FIG. 25 shows an exploded top-down view of a catheter securement system, according to an exemplary embodiment of the present disclosure;

FIG. 26 shows an exploded bottom-up view of a catheter securement system, according to an exemplary embodiment of the present disclosure;

FIG. 27 shows an exploded top-down view of a replacement top layer dressing of a dressing system, according to an exemplary embodiment of the present disclosure; and

FIG. 28 shows an exploded bottom-up view of a replacement top layer dressing of a dressing system, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

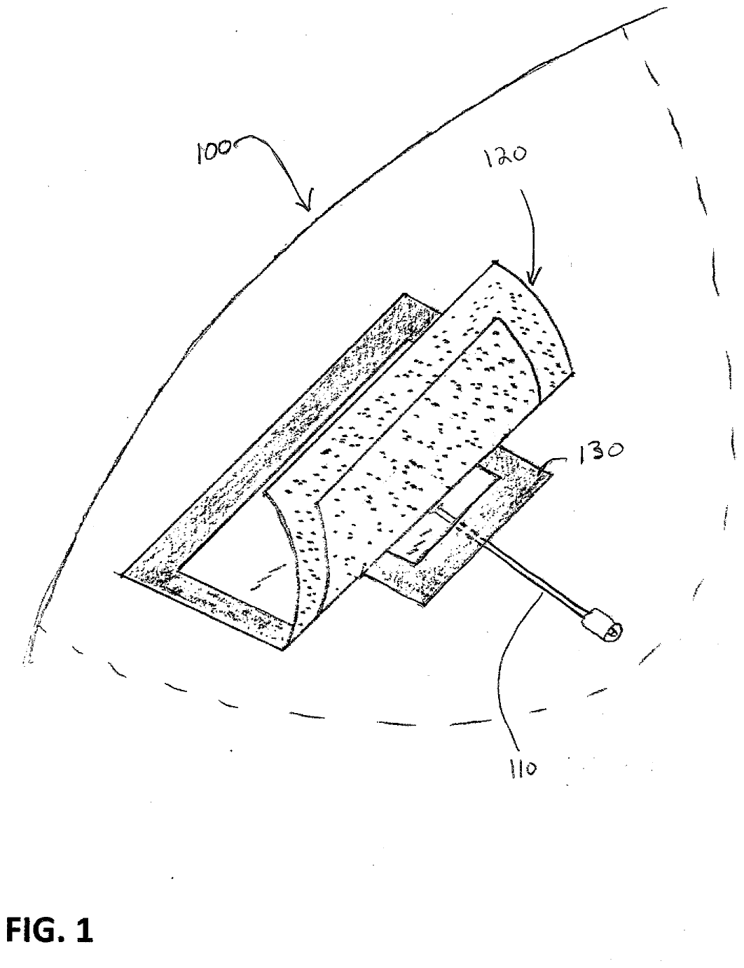

FIG. 1 shows a view of an exemplary dressing system 100 of the present disclosure, whereby the top layer dressing 120 is in the process of being removed from the bottom layer dressing 130, leaving the bottom layer dressing 130 behind as adhered in the patient's skin. A catheter 110 is also shown, whereby the point of catheter 110 entry into the patient's skin is covered by top layer dressing 120.

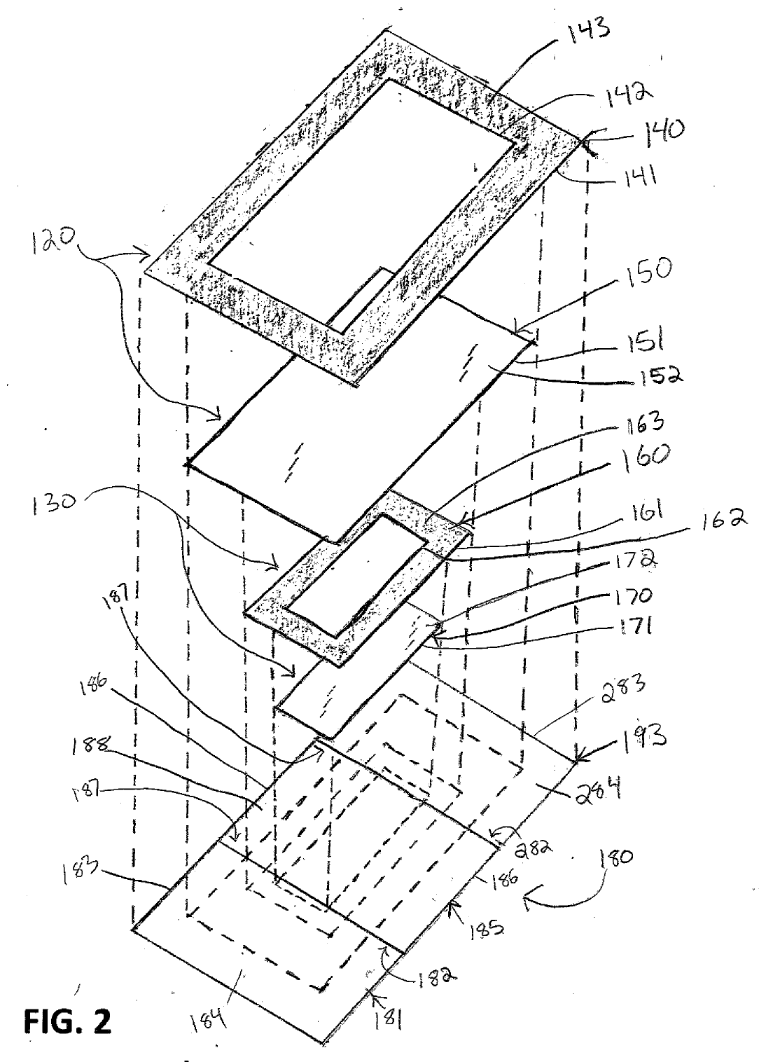

FIG. 2 shows an exploded top-down view of an exemplary dressing system 100 of the present disclosure. As shown therein, top layer dressing 120 has a top layer border area 140 comprising DM-2270 or another suitable dressing material, such as an elastic polyurethane non-woven material with medical grade adhesive on one side. Outer border 141 and inner border 142 are shown, with non-adhesive side 143 shown at a relative top of the figure. A top layer central area 150, comprising DM-4305 or another suitable dressing material such as a clear polyurethane film with a medical grade adhesive on one side, is also shown in FIG. 2. Peripheral border 151 is also shown, with non-adhesive side 152 shown on a relative top of said portion of top layer dressing 120. Bottom layer dressing 130 is shown relatively below top layer dressing 120, with bottom layer border area 160 comprising DM-2270 or another suitable dressing material, such as an elastic polyurethane non-woven material with medical grade adhesive on one side. Outer border 161 and inner border 162 is shown, with non-adhesive side 163 shown at a relative top of said portion of bottom layer dressing 130. A bottom layer central area 170, comprising DM-4305 or another suitable dressing material such as a clear polyurethane film with a medical grade adhesive on one side, is also shown in FIG. 2. Peripheral border 171 is also shown, with non-adhesive side 172 shown on a relative top of said portion of bottom layer dressing 130.

FIG. 2 also shows a carrier 180, with said carrier 180 cut into three pieces in an exemplary embodiment. As shown therein, carrier 180 has a lateral area 181, an inner border 182, a peripheral border 183, and a surface 184 of carrier 180 facing the adhesive portions of top layer dressing 120 and bottom layer dressing 130. A lateral area 193 of carrier 180 is also shown, with inner border 282, peripheral border 283, and surface 284 of carrier 180 facing the adhesive portions of top layer dressing 120 and bottom layer dressing 130. FIG. 2 also shows a central area 185 of carrier 180, with peripheral border 186, inner border 187, and a central surface 188 of carrier 188 facing the adhesive portions of top layer dressing 120 and bottom layer dressing 130.

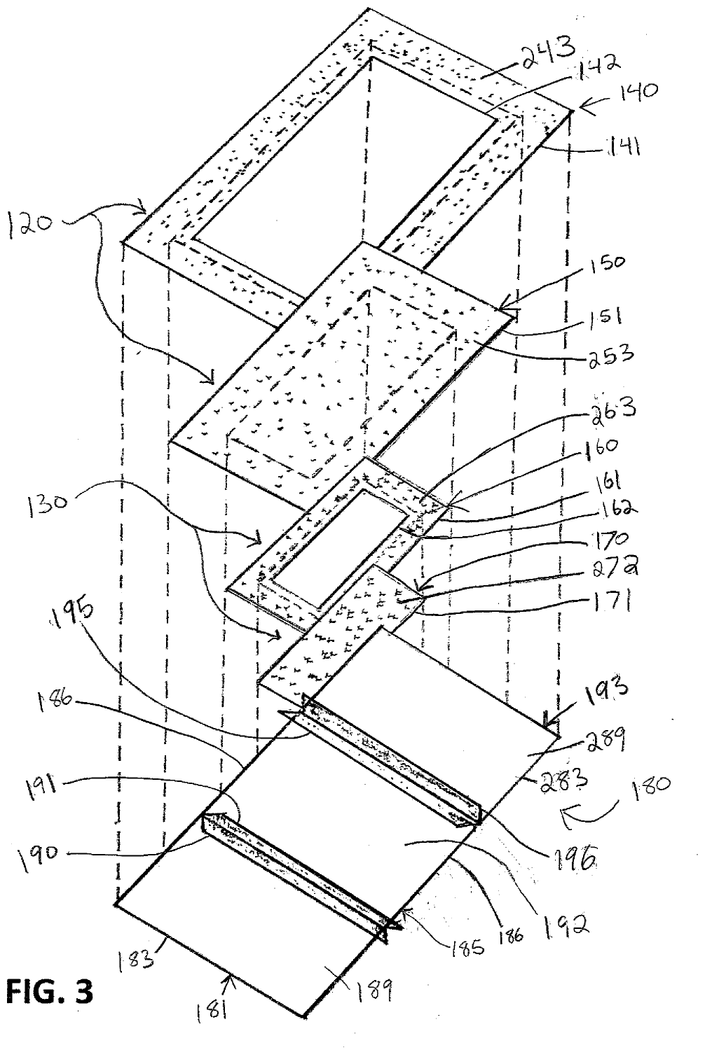

FIG. 3 shows an exploded bottom-up view of an exemplary dressing system 100 of the present disclosure, also with a three-piece carrier 180. As shown therein, top layer dressing 120 has a top layer border area 140 comprising DM-2270 or another suitable dressing material, such as an elastic polyurethane non-woven material with medical grade adhesive on one side. Outer border 141 and inner border 142 are shown, with adhesive side 243 shown at a relative top of the figure. A top layer central area 150, comprising DM-4305 or another suitable dressing material such as a clear polyurethane film with a medical grade adhesive on one side, is also shown in FIG. 3. Peripheral border 151 is also shown, with adhesive side 253 shown on a relative top of said portion of top layer dressing 120. Bottom layer dressing 130 is shown relatively below top layer dressing 120, with bottom layer border area 160 comprising DM-2270 or another suitable dressing material, such as an elastic polyurethane non-woven material with medical grade adhesive on one side. Outer border 161 and inner border 162 is shown, with adhesive side 263 shown at a relative top of said portion of bottom layer dressing 130. A bottom layer central area 170, comprising DM-4305 or another suitable dressing material such as a clear polyurethane film with a medical grade adhesive on one side, is also shown in FIG. 3. Peripheral border 171 is also shown, with adhesive side 272 shown on a relative top of said portion of bottom layer dressing 130.

FIG. 3 also shows a carrier 180, with said carrier 180 cut into three pieces in an exemplary embodiment. As shown therein, carrier 180 has a lateral area 181, a peripheral border 183 of lateral area 181, a pull tab 190 of said lateral area 181, and a lateral surface 189 of lateral area 181 side facing opposite the adhesive of top layer dressing 120 and bottom layer dressing 130. A lateral area 193 of carrier 180 is also shown, with a peripheral border 283 of lateral area 193, a pull tab 196 of said lateral area 193, and a lateral surface 289 of lateral area 193 side facing opposite the adhesive of top layer dressing 120 and bottom layer dressing 130. FIG. 3 also shows a central area 193 of carrier 180, with a peripheral border 186 of central area 193, pull tabs 191 and 195 of central area 185, and a central surface 192 of central area 185 side facing opposite the adhesive of top layer dressing 120 and bottom layer dressing 130.

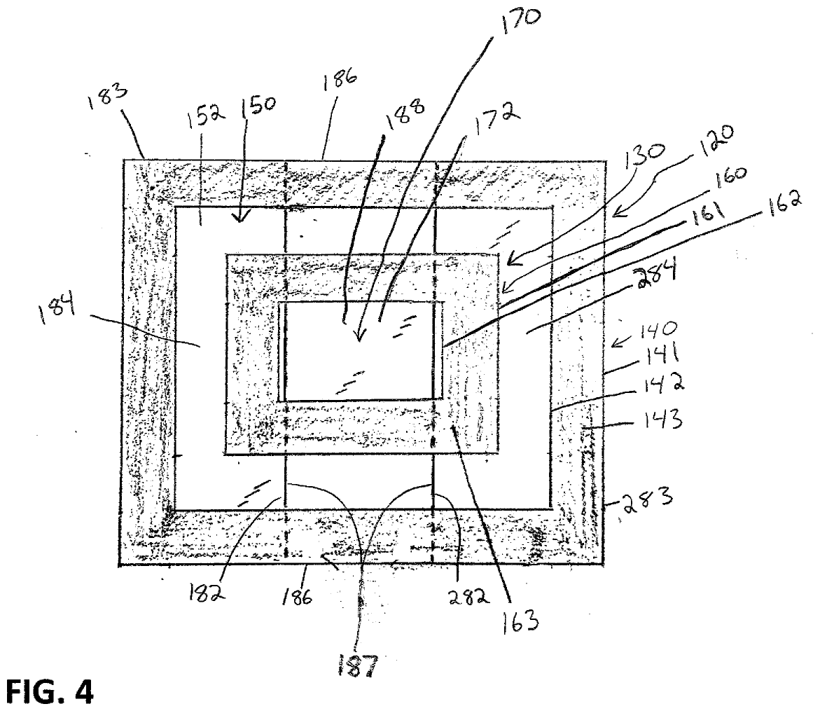

FIG. 4 shows a top-down view of an exemplary dressing system 100 of the present disclosure showing the relation of the three-piece carrier 180 to the top layer dressing 120 and the bottom layer dressing 130. The bottom layer dressing 130 can be seen through the top layer central area 150. Carrier borders 182 and 187 can be partially seen through the top layer central area 150. Carrier borders 182 and 187 may also be seen through top layer central area 150 and bottom layer central area 170.

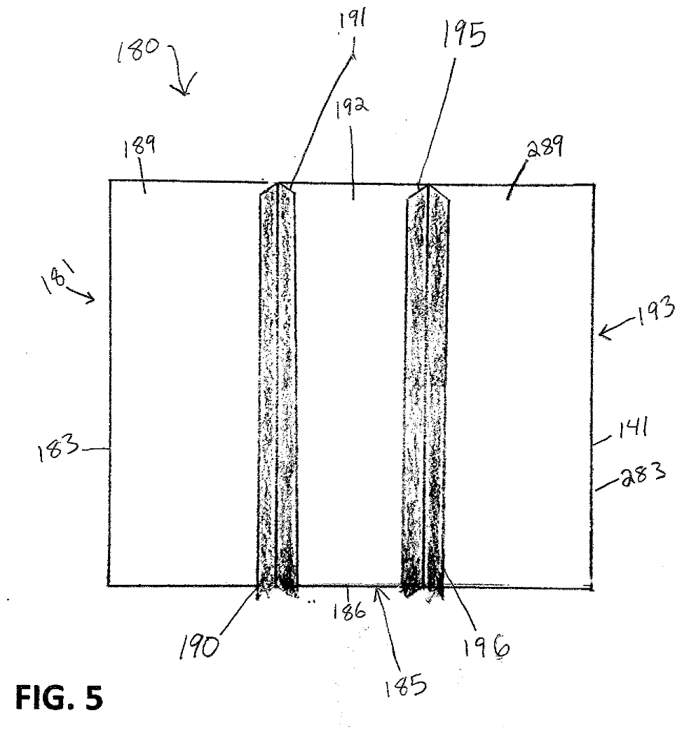

FIG. 5 shows a bottom view of an exemplary dressing system 100 of the present disclosure. As shown therein, carrier 180 has lateral area 181, lateral area 193, and central area 185, with pull tabs 191, 195 of central area shown, pull tab 190 of lateral area 181 shown, and pull tab 196 of lateral area 193 also shown.

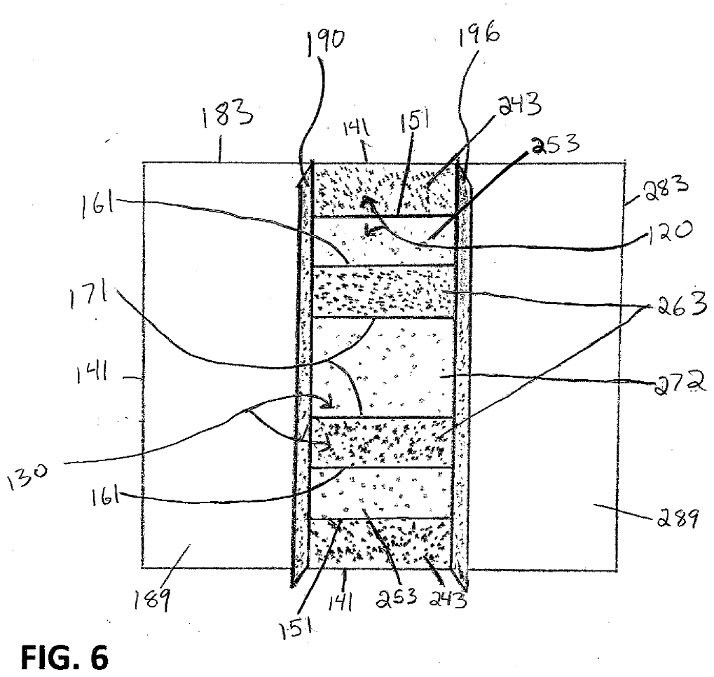

FIG. 6 shows a bottom view of an exemplary dressing system 100 of the present disclosure with central area 185 removed from said dressing system 100. Top layer dressing 120 and bottom layer dressing 130 can be partially seen in said figure.

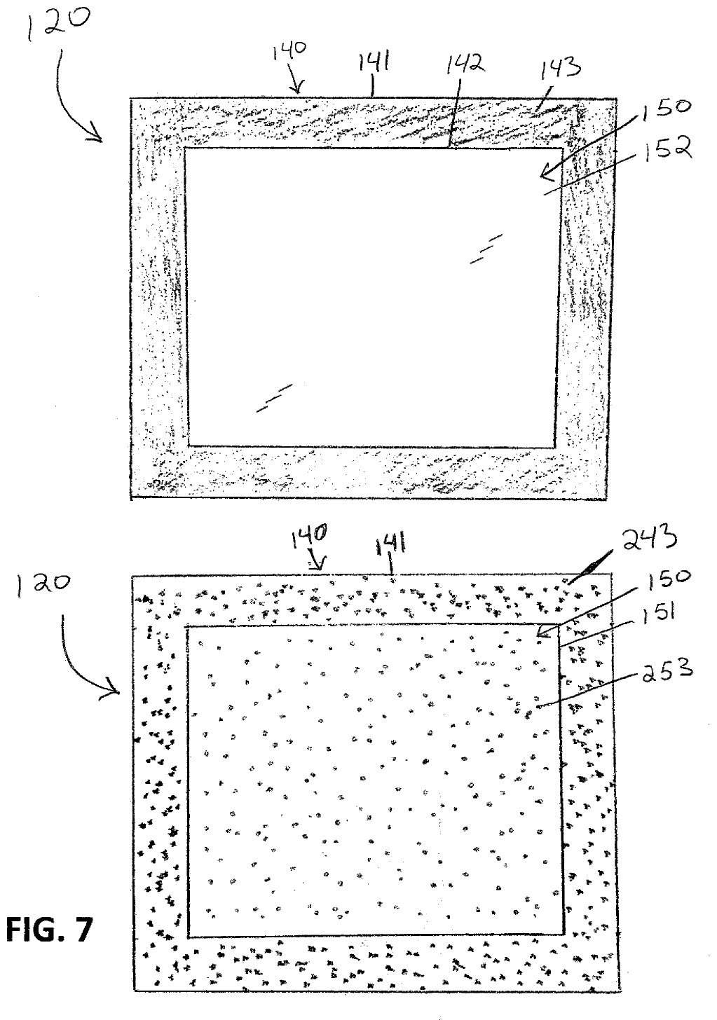

FIG. 7 shows a top view (upper portion of figure) and a bottom view (lower portion of figure) of an exemplary top layer dressing 120 of the present disclosure.

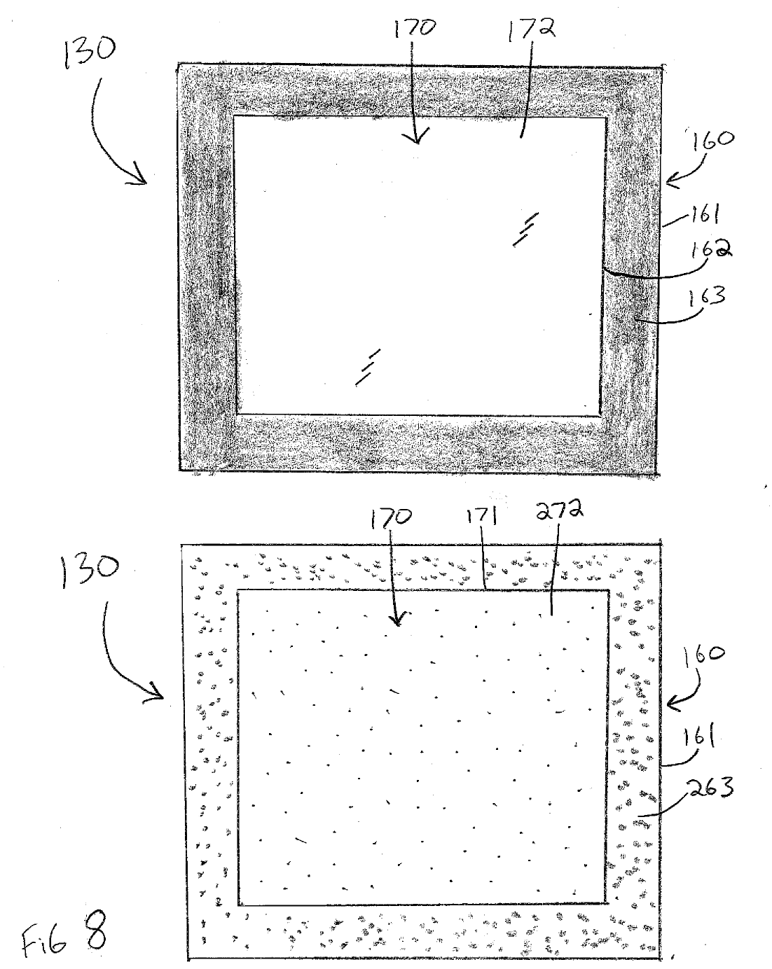

FIG. 8 shows a top view (upper portion of figure) and a bottom view (lower portion of figure) of an exemplary bottom layer dressing 130 of the present disclosure.

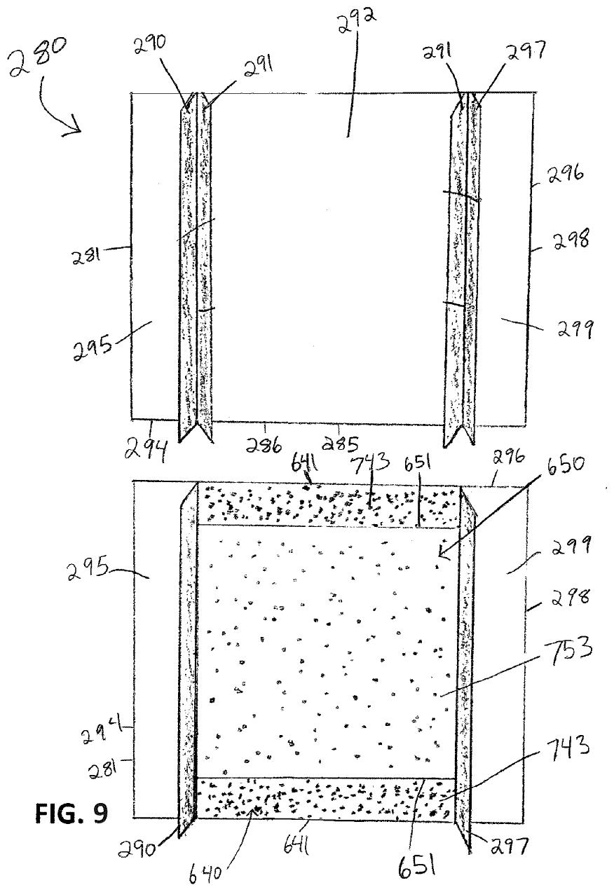

FIG. 9 shows bottom views of an exemplary dressing system 100 of the present disclosure, with central area 285 present (upper portion of figure) and removed (lower portion of figure). FIG. 9 shows a replacement top layer dressing 620 bottom view and carrier 280 cut into three pieces, with central area 285 removed, namely a bottom view showing relation to the top layer dressing 120. The relatively larger width of central area 285 as compare to lateral areas 281, 298 allows for bottom layer dressing 130 to be better visualized when placing a replacement top layer dressing 620.

As shown in FIG. 9, carrier 280 is cut into three pieces, with lateral area 281, lateral area 285, and central area 285 shown therein. A peripheral border 294 of lateral area 281 is shown, with pull tab 290 of lateral area 281 and carrier surface 295 facing the opposite of adhesive of 743 and 753. A peripheral border 296 of lateral area 298 is also shown, with pull tab 297 of lateral area 298 and carrier surface 299 facing the opposite of adhesive of 743 and 753. A peripheral border 286 of central area 285 is also shown, with pull tabs 291 of central area 285 and central surface 292 of the carrier side facing the opposite of adhesive of 743 and 753.

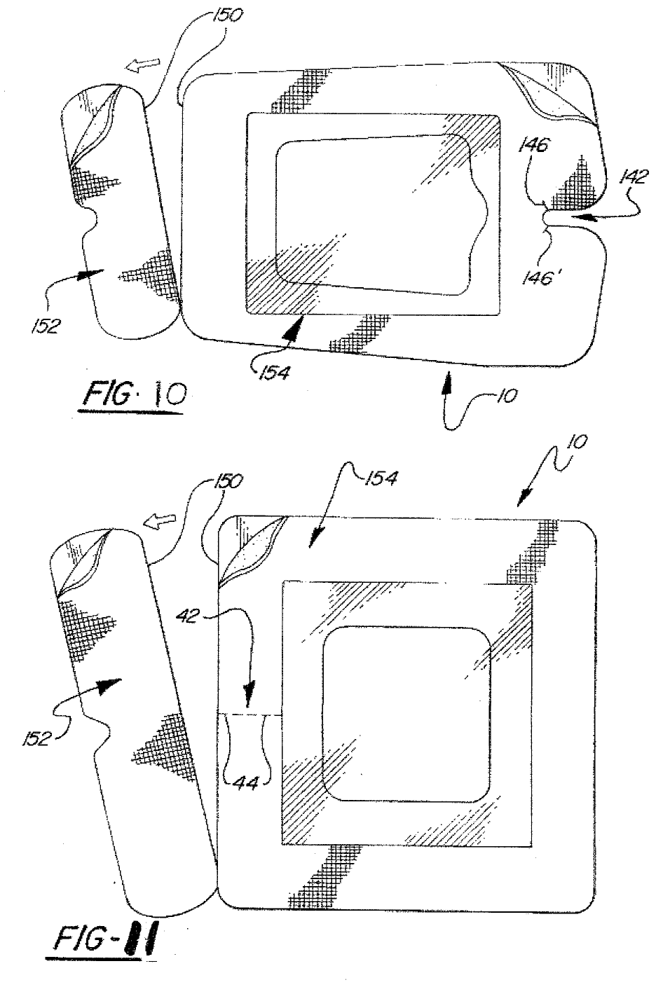

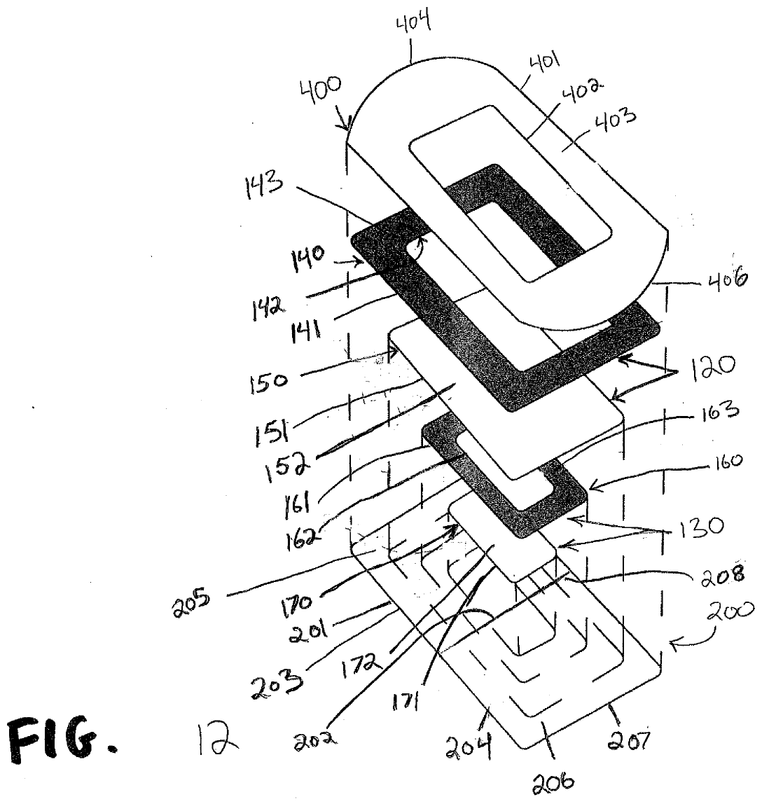

Dressing systems of the present disclosure can also comprise the use of a separable portion of a tape layer to be separated from the dressing portion, such as disclosed as element 152 in U.S. Pat. No. 6,124,521. FIGS. 3 and 4 of U.S. Pat. No. 6,124,521 are incorporated herein as FIGS. 10 and 11.

FIG. 12 shows an exploded top-down view of an exemplary dressing system 100 of the present disclosure. Window frame 400 is shown therein with carrier 200 cut in half (so a two-piece carrier 200). Window frame 400 helps the dressing system 100 replacement top layer or bottom layer dressing maintain its shape while being applied, noting that it is removed immediately after application. A peripheral border 401 of window frame 400 is shown, with portions 404, 406 of window frame 400 extending beyond one or more edges of the top layer dressing 120 to provide a surface to hold onto while applying an exemplary dressing system 100 of the present disclosure. Inner border 404 is also shown, with surface 403 facing away from the replacement top layer or bottom layer of said dressing.

Carrier 200 is shown cut in two, with carrier piece 203 having a surface 205 facing the adhesive, a peripheral border 201, and an inner border 202, and with carrier piece 204 having a surface 206 facing the adhesive, a peripheral border 207, and an inner border 208.

FIG. 13 shows an exploded bottom-up view of an exemplary dressing system 100 of the present disclosure. Window frame 400 is shown therein with carrier 200 cut in half (so a two-piece carrier 200). Window frame 400 helps the dressing system 100 replacement top layer or bottom layer dressing maintain its shape while being applied, noting that it is removed immediately after application. A peripheral border 401 of window frame 400 is shown, with portions 404, 406 of window frame 400 extending beyond one or more edges of the top layer dressing 120 to provide a surface to hold onto while applying an exemplary dressing system 100 of the present disclosure. Inner border 404 is also shown, with surface 405 facing the replacement top layer or bottom layer of said dressing.

Carrier 200 is shown cut in two, with carrier piece 203 having a peripheral border 201, an inner border 202, a surface 210 facing opposite the adhesive, and a pull tab 211, and with carrier piece 204 having a peripheral border 207, an inner border 208, a surface 209 facing opposite the adhesive, and a pull tab 212.

FIG. 14 shows an exploded top-down view of a catheter securement system of the present disclosure. As shown therein, catheter securement system has a window frame 6 and a carrier 10 cut in two. Window frame 6 having a peripheral border 7, a surface 8 opposite the catheter securement 1, and a cut out 9 for a catheter is shown therein. Portions 24, 25 of window frame 6 extend beyond the border of the catheter securement 1 to provide a surface to hold onto while placing the catheter securement 1. Carrier securement 1 is shown therein, with peripheral border 2, a surface 3 opposite the adhesive side, and a cut out 4 for a catheter. Carrier 10 is shown with carrier pieces 11, 12, with carrier piece 11 having a peripheral border 16, an inner border 18, and a surface 17 facing the adhesive, and with carrier piece 12 having a peripheral border 13, an inner border 15, and a surface 14 facing the adhesive.

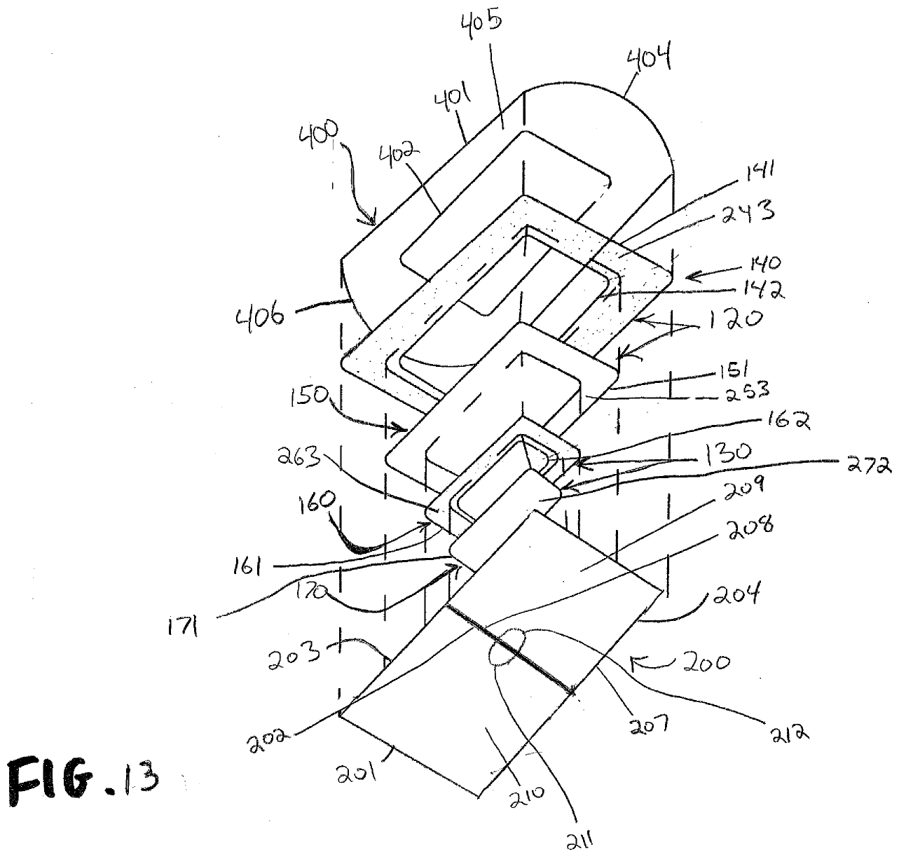

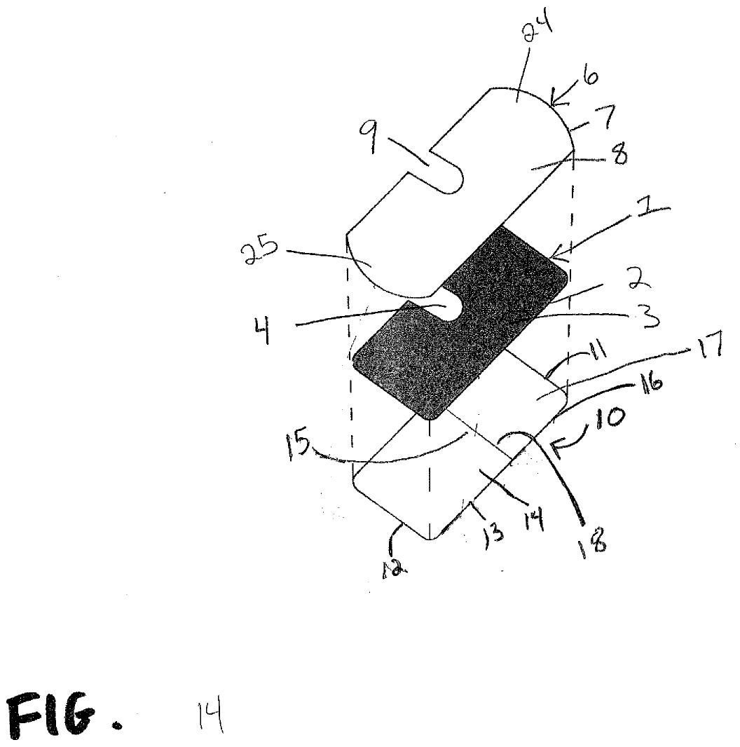

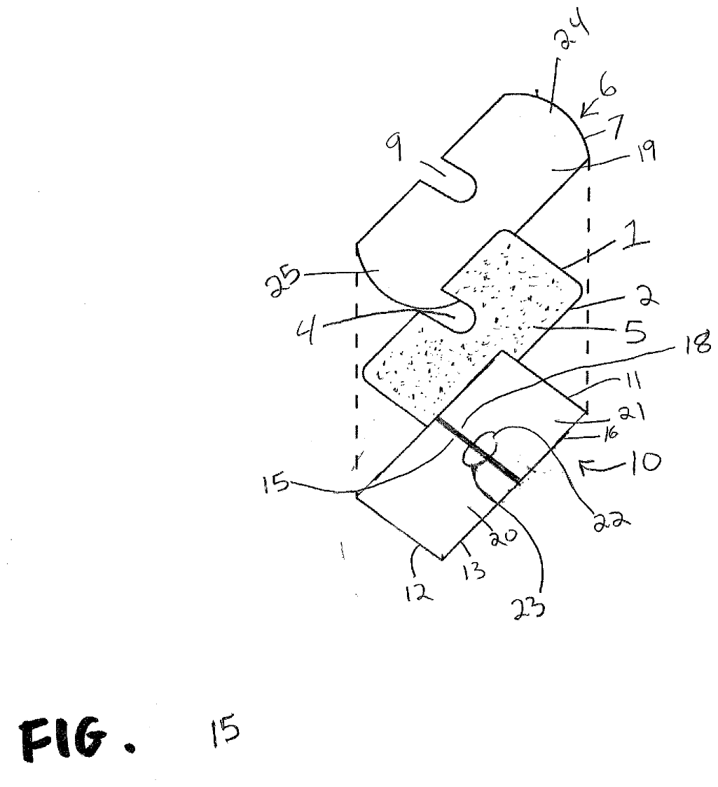

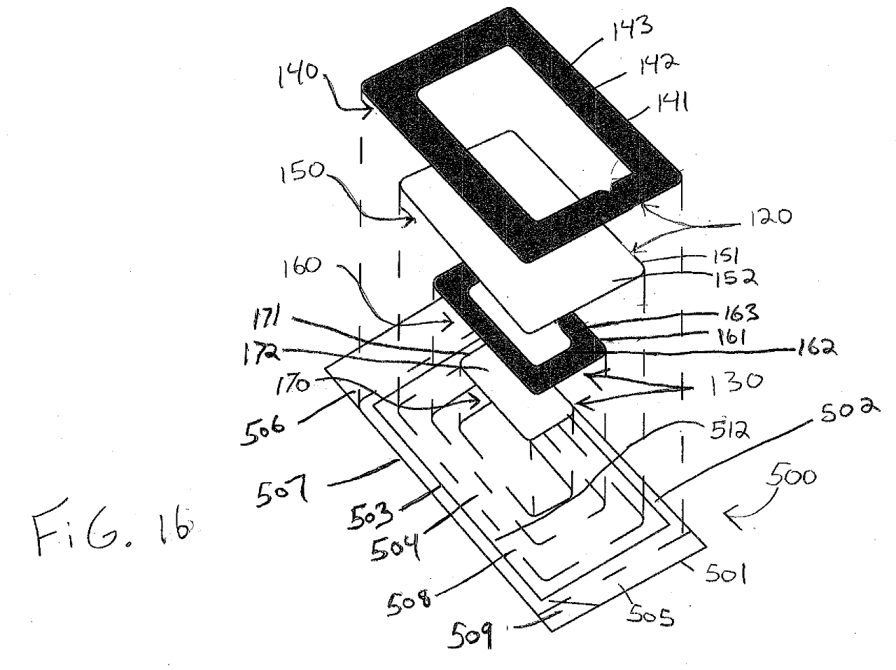

FIG. 15 shows an exploded bottom-up view of a catheter securement system of the present disclosure. As shown therein, catheter securement system has a window frame 6 and a carrier 10 cut in two. Window frame 6 having a peripheral border 7, a surface 19 facing the catheter securement 1, and a cut out 9 for a catheter is shown therein. Portions 24, 25 of window frame 6 extend beyond the border of the catheter securement 1 to provide a surface to hold onto while placing the catheter securement 1. Carrier securement 1 is shown therein, with peripheral border 2, a surface 5 adhesive side, and a cut out 4 for a catheter. Carrier 10 is shown with carrier pieces 11, 12, with carrier piece 11 having a peripheral border 16, an inner border 18, a surface 21 facing opposite the adhesive, and a pull tab 22, and with carrier piece 12 having a peripheral border 13, an inner border 15, a surface 20 facing opposite the adhesive, and a pull tab 23.

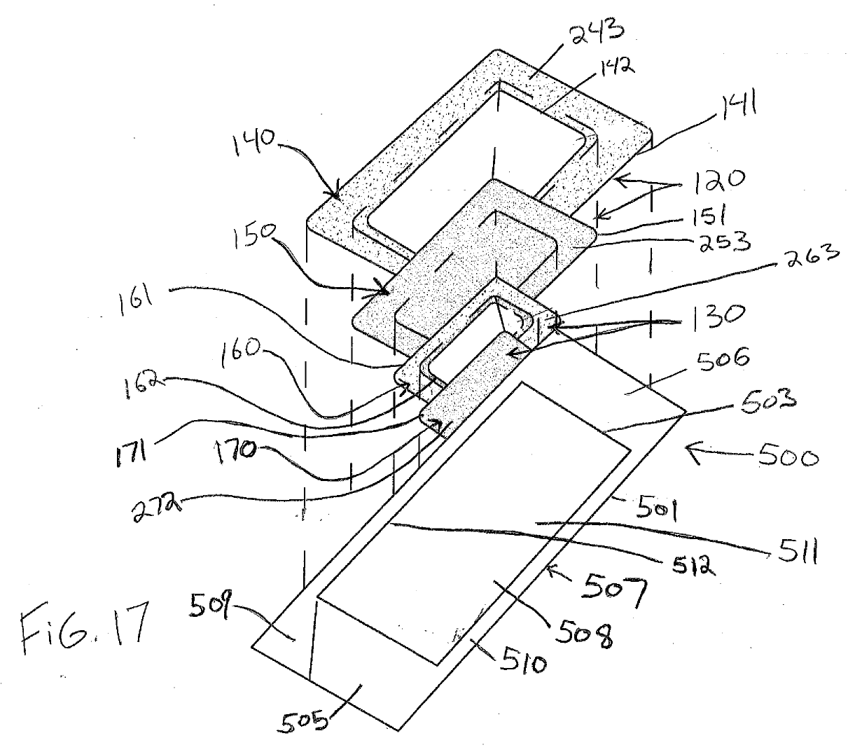

FIG. 16 shows an exploded top-down view of an exemplary dressing system 100 of the present disclosure. A two-piece carrier 500 having a central piece 508 and a peripheral piece 507 is shown therein. Peripheral piece 507 of carrier 500 has a peripheral border 501, a central border 512, a peripheral surface 502 facing the adhesive of an outer perimeter of a top layer adhesive 243, a pull tab 509, and handles 505, 506. Central piece 508 of carrier 500 has a peripheral border 503 and a central surface 504 facing the adhesive covering the inner periphery of a top layer adhesive covering an inner periphery of a top layer adhesive 243 and completely covering a top layer central area 253.

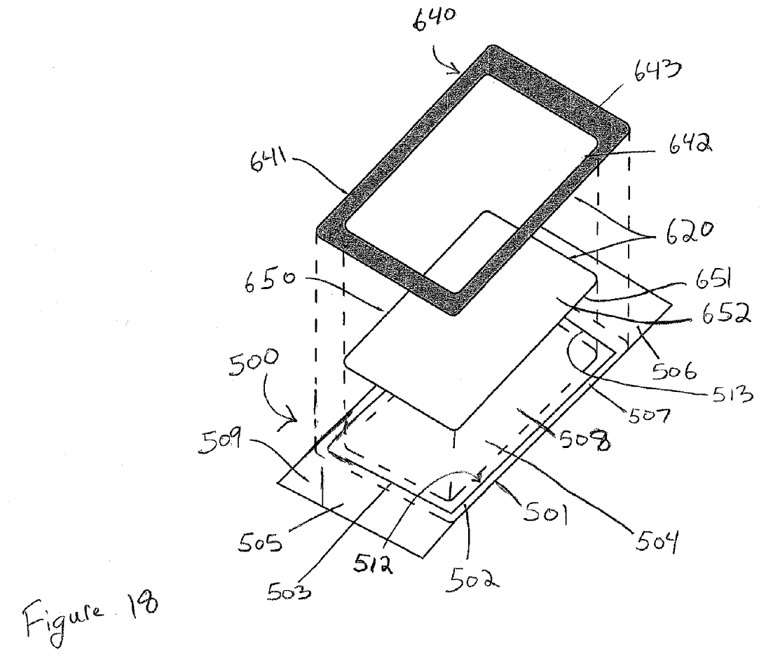

FIG. 17 shows an exploded bottom-up view of an exemplary dressing system 100 of the present disclosure. A two-piece carrier 500 having a central piece 508 and a peripheral piece 507 is shown therein. Peripheral piece 507 of carrier 500 has a peripheral border 501, a central border 512, a peripheral surface 510 facing opposite the adhesive of an outer perimeter of a top layer adhesive 243, a pull tab 509, and handles 505, 506. Central piece 508 of carrier 500 has a peripheral border 503 and a central surface 511 facing opposite the adhesive covering the inner periphery of a top layer adhesive covering an inner periphery of a top layer adhesive 243 and completely covering a top layer central area 253.

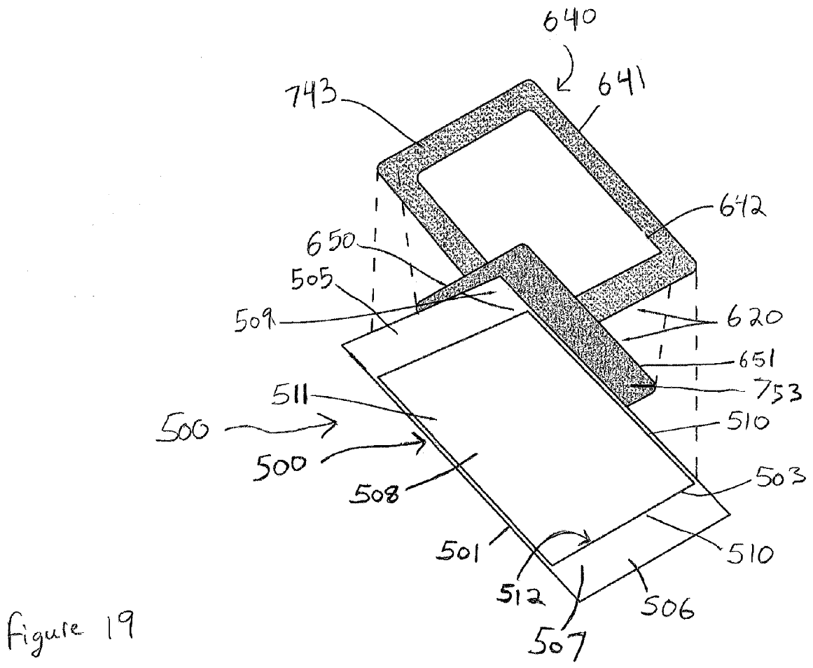

FIG. 18 shows an exploded top-down view of a replacement top layer dressing 620 of an exemplary dressing system 100 of the present disclosure, such as one using a two-piece carrier 500. As shown therein, replacement top layer dressing 620 has a top layer border area 640 comprising DM-2270 or another suitable dressing material such as an elastic polyurethane non-woven material with medical grade adhesive on one side, an outer border 641, an inner border 642, and a non-adhesive side 643. Replacement top layer dressing 620 also has a top layer central area 650 comprising DM-4305 or another suitable dressing material such as a clear polyurethane film with medical grade adhesive on one side, a peripheral border 651, and a non-adhesive side 652. A two-piece carrier 500 can be used, comprising a peripheral piece 501 having a peripheral border 501, a central border 512, a peripheral surface 502 facing the adhesive of outer perimeter of top layer adhesive 743, a pull tab 509, and handles 505, 506. Central piece 508 of carrier 500 has a peripheral border and a central surface 504 facing the adhesive covering the inner periphery of top layer adhesive 743 and completely covering top layer central area 753.

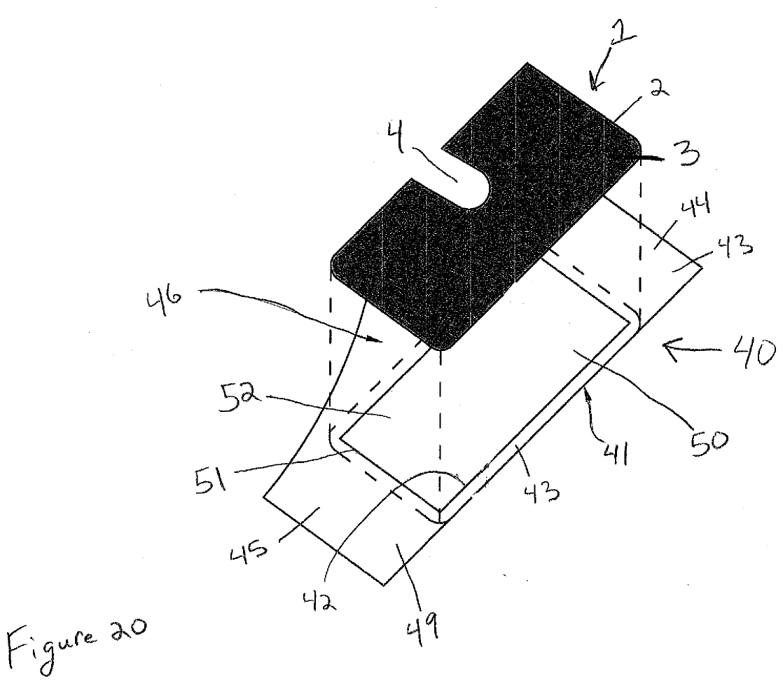

FIG. 19 shows an exploded bottom-up view of a replacement top layer dressing 620 of an exemplary dressing system 100 of the present disclosure, such as one using a two-piece carrier 500. As shown therein, replacement top layer dressing 620 has a top layer border area 640 comprising DM-2270 or another suitable dressing material such as an elastic polyurethane non-woven material with medical grade adhesive on one side, an outer border 641, an inner border 642, and an adhesive side 743. Replacement top layer dressing 620 also has a top layer central area 650 comprising DM-4305 or another suitable dressing material such as a clear polyurethane film with medical grade adhesive on one side, a peripheral border 651, and an adhesive side 753. A two-piece carrier 500 can be used, comprising a peripheral piece 501 having a peripheral border 501, a central border 512, a 510 peripheral area facing opposite the adhesive of outer perimeter of top layer 743, a pull tab 509, and handles 505, 506. Central piece 508 of carrier 500 has a peripheral border and a 511 central surface facing opposite the adhesive.

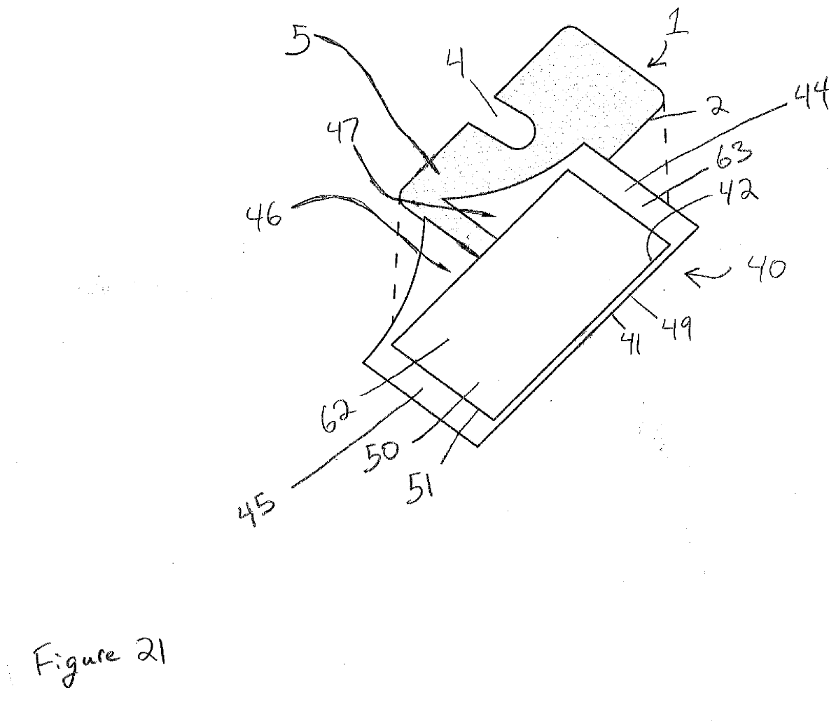

FIG. 20 shows an exploded top-down view of a catheter securement system of the present disclosure, such as one that uses a two-piece carrier 40. As shown therein, catheter securement system has an outer border 2, a surface 3 opposite the adhesive side, and a cut out 4 for a catheter. Carrier 40, as shown in FIG. 20, has a peripheral piece 49 and a central piece 50. Peripheral piece 49 of carrier 40 has a peripheral border 41, an inner border 42, a surface 43 facing the adhesive, handles 44, 45, and a pull tab 46. Central piece 50 of carrier 40 has a peripheral border 51 and a surface 52 facing the adhesive.

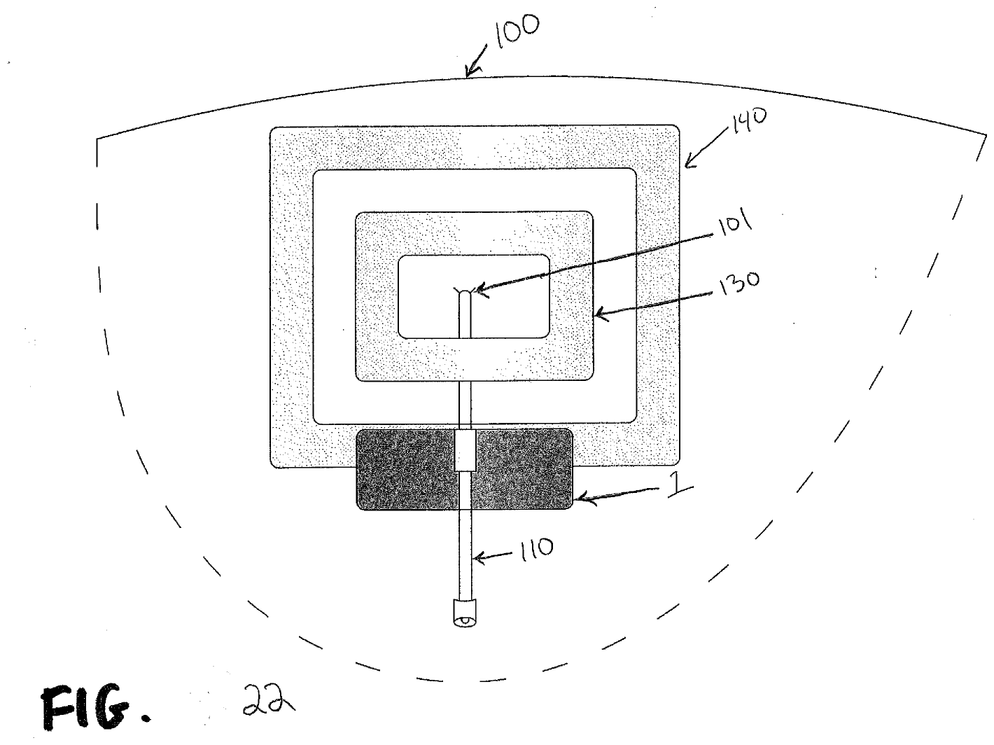

FIG. 21 shows an exploded bottom-up view of a catheter securement system of the present disclosure, such as one that uses a two-piece carrier 40. As shown therein, catheter securement system has an outer border 2, a surface 5 adhesive side, and a cut out 4 for a catheter. Carrier 40, as shown in FIG. 20, has a peripheral piece 49 and a central piece 50. Peripheral piece 49 of carrier 40 has a peripheral border 41, an inner border 42, a surface 63 facing opposite the adhesive, handles 44, 45, and a pull tab 46. Central piece 50 of carrier 40 has a peripheral border 51 and a surface area 62 facing opposite the adhesive.

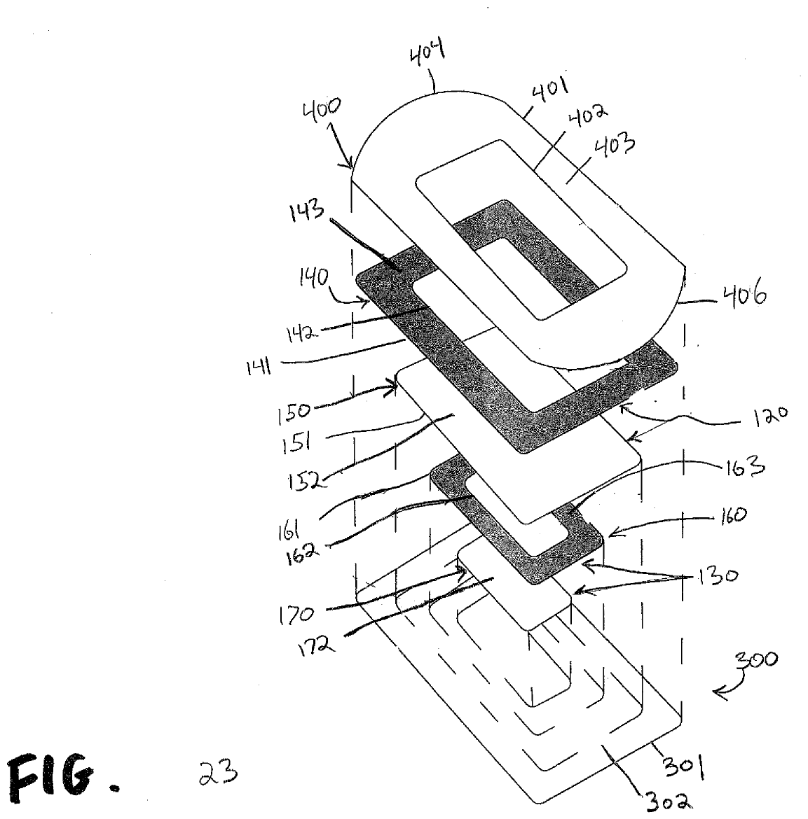

FIG. 22 shows a top-down view of an exemplary dressing system 100 being used to cover an insertion site 101 of an indwelling catheter 110 into the skin. Catheter securement 1 is holding down the peripheral area 143 of top layer 140 over exiting catheter 110 to prevent dislodgment and dressing system 100 disruption.

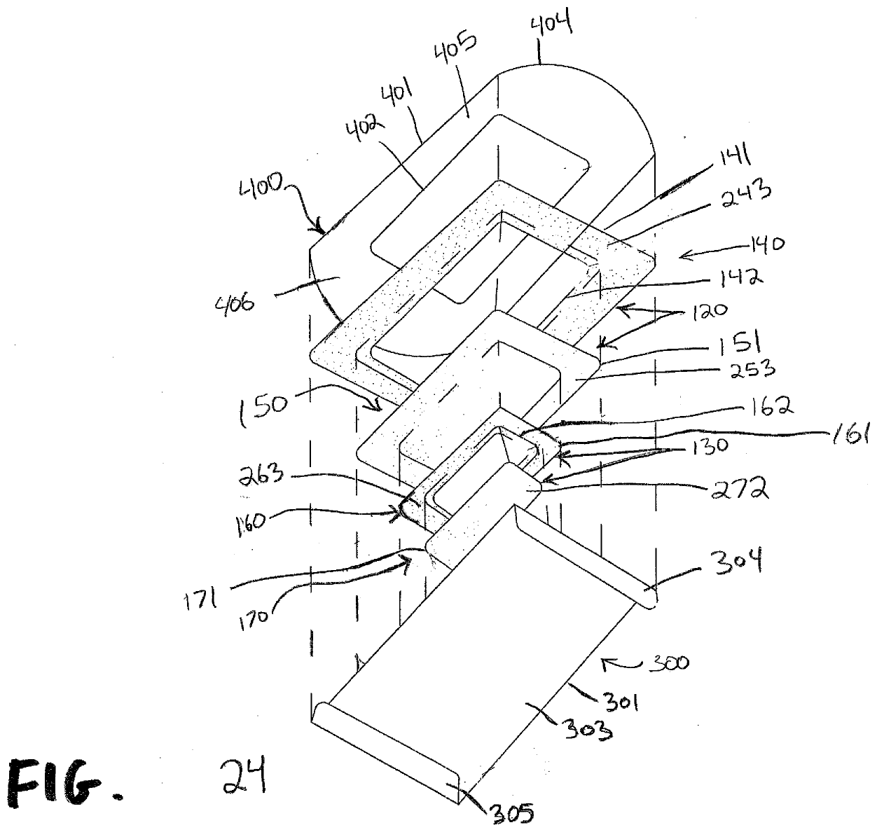

FIG. 23 shows an exploded top-down view of a dressing system 100, similar to that as shown in FIG. 23, with window frame 400 and a one-piece carrier 300. Carrier 300 has a carrier area 302 facing the adhesive and a peripheral border 301.

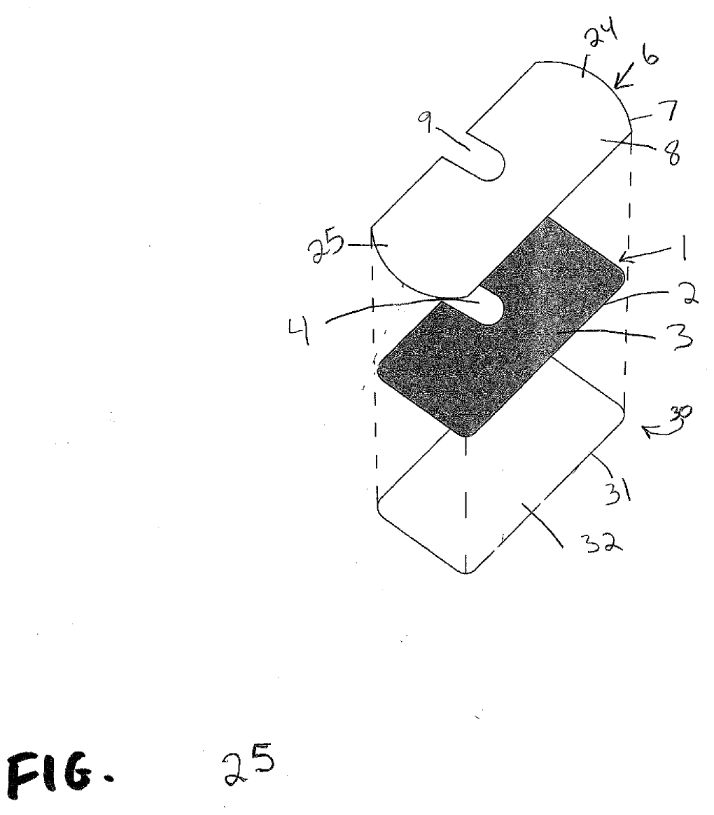

FIG. 24 shows an exploded bottom-up view of a dressing system 100, similar to that as shown in FIG. 24, with window frame 400 and a one-piece carrier 300. Carrier 300 has a carrier area 303 facing opposite the adhesive, a peripheral border, and pull tabs 304, 305.

FIG. 25 shows an exploded top-down view of a catheter securement system of the present disclosure, similar to that as shown in FIG. 14, with a window frame 6 and a one-piece carrier 30. Carrier 30 has a peripheral border 31 and a one-piece carrier area 32 facing the adhesive.

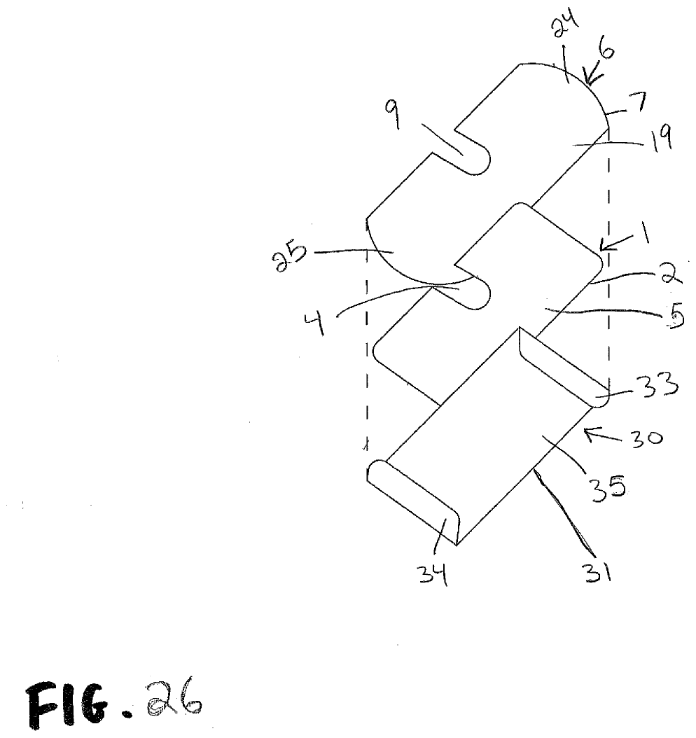

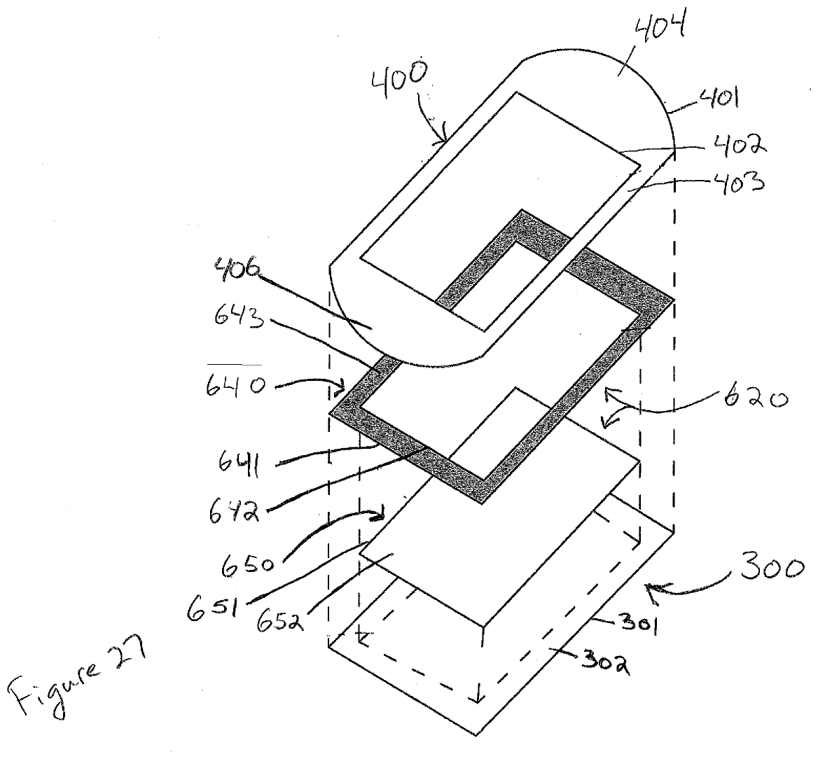

FIG. 26 shows an exploded bottom-up view of a catheter securement system of the present disclosure, similar to that as shown in FIG. 14, with a window frame 6 and a one-piece carrier 30. Carrier 30 has a peripheral border 31, pull tabs 34, 35, and a one-piece carrier 35 facing opposite the adhesive.

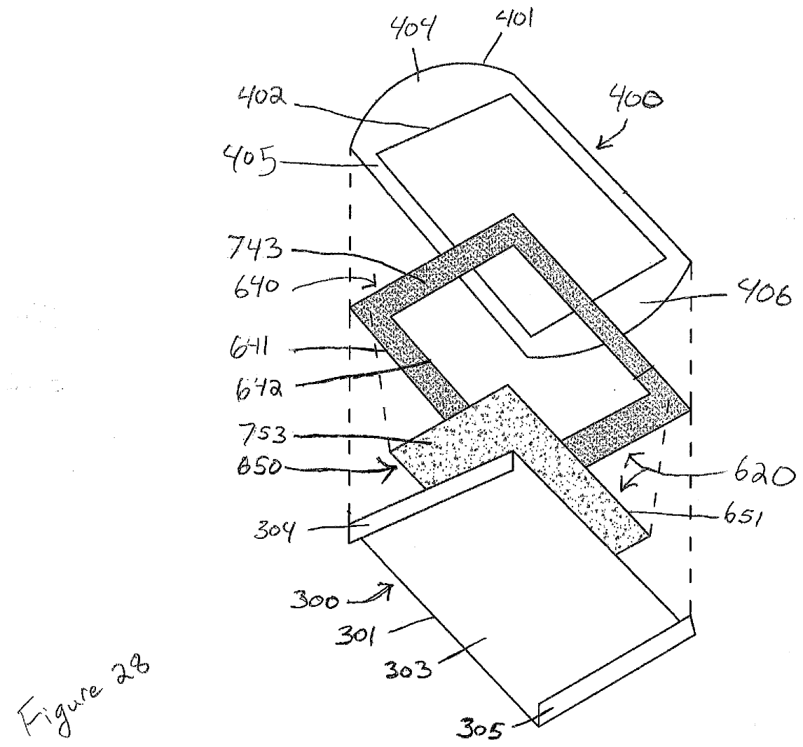

FIG. 27 shows an exploded top-down view of a replacement top layer dressing 620 of an exemplary dressing system 100 of the present disclosure, similar to that shown in FIG. 18, but using a one-piece carrier 300. As shown therein, carrier 300 has a carrier area 302 facing the adhesive and a peripheral border 301.

FIG. 28 shows an exploded bottom-up view of a replacement top layer dressing 620 of an exemplary dressing system 100 of the present disclosure, similar to that shown in FIG. 19, but using a one-piece carrier 300. As shown therein, carrier 300 has a carrier area 303 facing opposite the adhesive, pull tabs 304, 305, and a peripheral border 301.

The dressing systems 100 of the present disclosure are not limited to the materials listed and may be comprised of any suitable dressing material. A catheter securement device 1, such as DM-2270 may be placed after a dressing system of the present disclosure is placed. A catheter securement device 1 is placed under an intravenous or pain block catheter and over a top layer border area DM-2270 of a dressing system noted herein. A catheter securement device 1 has a length and width approximately the size of one side of top layer border area DM-2270.

As noted in the figures, a carrier is used to support various layers of an exemplary dressing system 100 of the present disclosure. As shown therein, various layers can have a central area of DM-4305, which is an exemplary thin polyurethane (transparent window) film distributed by Global Biomedical Technologies LLC of Greensboro, GA coated on one side with a low tack pattern coated, medical grade pressure sensitive acrylic adhesive to create a highly breathable, transparent window dressing with the adhesive side of bottom layer facing towards the skin. Said base bottom layers of an exemplary dressing system 100 further comprises a border area of DM-2270, which is a film also distributed by Global Biomedical Technologies LLC of Greensboro, GA consisting of a nonwoven polyurethane fabric coated on one side with a medical grade, pressure sensitive acrylic adhesive blended with a proprietary additive, surrounding the DM-4305. Bottom layer border area DM-2270 is adhered to the periphery of bottom layer central area DM-4305 and skin.

The top layer dressing 130 is similar to bottom layer dressing 120 but larger than bottom layer dressing 120. Top layer dressing 130 has a central area of DM-4305 with an adhesive side facing towards the bottom layer dressing 120 and skin. The central area of top layer dressing 13—DM-4305 completely surrounds the bottom layer dressing 120. Top layer dressing 130 further comprises a border area DM-2270 surrounding top layer DM-4305 central area. Top layer dressing 130 border area DM-2270 is adhered to the periphery of top layer central area DM-4305 and the skin.

The dressing systems of the present disclosure may be applied as one unit design. Both the top layer and bottom layer come pre-assembled on a common carrier covering the adhesive. The top layer may have a removable window frame opposite the carrier to allow the dressing to maintain shape during application. The carrier is cut into three pieces creating two lateral areas and a central area. The central area of the carrier is covering the central area DM-4305 of the bottom layer. The central area of the carrier also covers the bottom layer border area DM-2270 above and below the bottom layer central area DM-4305. The central area of the carrier does not cover the bottom layer border area DM-2270 that is bilateral to bottom layer central area DM-4305. The central area of the carrier also covers the top layer area of DM-4305 and DM-2270 that over hangs above and below the bottom layer. The central area of the carrier does not cover the top layer area of DM-4305 and DM-2270 that over hangs the bottom layer bilaterally. The lateral areas cover the bottom layer border area DM-2270 that is bilateral to bottom layer central area DM-4305 and top layer area of DM-4305 and DM-2270 that over hangs the bottom layer bilaterally.

During application the central carrier area is removed first, allowing the two lateral areas of the carrier to hold the bottom layer onto the top layer and preventing separation during application. The dressing is applied to the wound then the lateral areas of the carrier are removed and the remaining dressing is completely adhered. The window frame is removed last if utilized.

Alternately, the carrier of a dressing of the present disclosure built as one unit may be not be cut allowing it to be removed as one solid piece. The window frame may overhang the top layer bilaterally creating an area to hold on to while applying the dressing system as a single unit.

Another alternate carrier may be cut into two equal pieces. The dressing built as one unit would be applied by partially removing both pieces equally, adhering over the wound before removing the carriers completely and adhering the dressing completely.

Another alternate carrier may be cut into two pieces. A central area carrier covering DM-4305 of bottom layer, DM-2270 of bottom layer, and inner periphery of DM-2270 of top layer. An outer peripheral carrier area can cover the outer periphery of DM-2270 top layer. The outer peripheral carrier area may extend past the outer periphery of DM-2270 top layer. This extension area provides a surface to hold onto (namely a grip portion) while placing the dressing on a patient.

The replacement top layer is the same material and design as the top layer and is different in that it has a carrier covering the adhesive of both DM-2270 and DM-4305 and may also have a removable window frame opposite the carrier to allow the dressing to maintain shape during application. The carrier of the replacement top layer is cut into three pieces creating two lateral areas and a central area. The central area of the replacement top layer carrier is covering the central area DM-4305 of the replacement top layer. The central area of the replacement layer carrier also covers the replacement top layer border area DM-2270 above and below the replacement top layer central area DM-4305. The central area of the replacement top layer carrier does not cover the replacement top layer border area DM-2270 that is bilateral to replacement top layer central area DM-4305. The replacement top layer carrier lateral areas cover the replacement top layer border area DM-2270 that is bilateral to the replacement top layer central area DM-4305. When placing the replacement top layer, the central area carrier is removed. This allows the view of an already in place bottom layer thru the replacement top layer central area DM-4305. The replacement top layer central area DM-4305 acts like and window to visualize the bottom layer border area DM-2270. Bottom layer border area DM-2270 acts like a guide in placing replacement top layer dressing centrally over the bottom layer to prevent replacement top layer border DM-2270 adhesive from contacting bottom layer border area DM-2270. This is important as central area DM-4305 adhesive of top layer and replacement top layer is removable from bottom layer border area DM-2270 top side opposite from the adhesive and bottom layer central area DM-4305 top side opposite from the adhesive. This visualization of the bottom layer border DM-2270 thru replacement top layer central area DM-4305 prevents replacement top layer border DM-2270 from contacting bottom layer border DM-2270 which may cause the dressing to fail during replacement top layer removal from the bottom layer.

Alternately, the replacement top layer carrier may be not be cut allowing it to be removed as one solid piece. The window frame of the replacement layer may overhang the replacement top layer bilaterally creating an area to hold on to while visualizing the bottom layer border DM-2270 thru replacement top layer DM-4305 and applying replacement top layer over a bottom layer and wound.

Another alternate replacement top layer carrier may be cut into two equal pieces. The replacement top layer would be applied by partially removing both carrier pieces equally, visualizing the bottom layer border DM-2270 thru replacement top layer DM-4305 and adhering over the bottom layer and wound before removing the carriers completely and adhering the replacement top layer completely.

Another alternate replacement top layer carrier may be cut into two pieces. A central area carrier covering DM-4305 and onto the inner periphery of DM-2270. An outer peripheral carrier area can cover the outer periphery of DM-2270. The outer peripheral carrier area may extend past the outer periphery of DM-2270. This extension area provides a surface to hold onto (namely a grip portion) while placing the dressing on a patient.

Dressing systems 100 of the present disclosure may be applied in a two-step process in which bottom layer dressing 120 is applied first followed by a replacement top layer dressing 130 as described. The bottom layer dressing 120 may also have a removable window frame opposite the carrier to allow the dressing to maintain shape during application. The bottom layer dressing 120 applied as the first step has a carrier cut into two equal pieces and applied by partially removing both pieces equally, visualizing the wound thru bottom layer central area DM-4305 and adhering over the wound before removing the carriers completely and adhering the bottom layer completely.

Alternatively, the bottom layer dressing 120 carrier may not be cut allowing it to be removed as one solid piece. The window frame of the bottom layer dressing 120 may overhang the bottom layer dressing 120 bilaterally creating an area to hold on to when placing the bottom layer dressing 120 on while visualizing the wound, intravenous device, or pain block catheter thru central area DM-4305.

The dressing system may be referred to as “2VIEW.”

While various embodiments of devices, systems, and methods of preparing and using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A dressing system, comprising:
a top layer comprising a first top layer element and a second top layer element, the first top layer element comprising a central area of a transparent window film having a first adhesive surrounded by the second top layer element comprising a border area of a polyurethane fabric having the first adhesive or a second adhesive;
a bottom layer comprising a first bottom layer element and a second bottom layer element, the first bottom layer element comprising a second central area of the transparent window film having the first adhesive surrounded by the second bottom layer element comprising a border area of the polyurethane fabric having the first adhesive or the second adhesive; and
a carrier configured to engage the top layer and the bottom layer.

2. The dressing system of claim 1, wherein the bottom layer is sized and shaped to be smaller than the central area of the top layer element.

3. The dressing system of claim 1, wherein the bottom layer is completely visible within the central area of the top layer element.

4. The dressing system of claim 1, wherein the first adhesive comprises a low-tack adhesive applied to a lower surface of the first top layer element in a pattern.

5. The dressing system of claim 1, wherein the second adhesive comprises a pressure sensitive adhesive blended with an adhesive additive.

6. The dressing system of claim 1, wherein the carrier comprises a middle portion and two side portions.

7. The dressing system of claim 1, wherein the carrier comprises two pull tabs positioned thereon, each pull tab adjacent a middle portion and a side portion of the carrier.

8. The dressing system of claim 1, wherein the carrier comprises a peripheral piece and a central piece located within the peripheral piece.

9. The dressing system of claim 8, wherein the peripheral piece comprises one pull tab.

10. The dressing system of claim 8, wherein the peripheral piece comprises two handles.

11. The dressing system of claim 8, wherein the peripheral piece comprises two pull tabs.

12. The dressing system of claim 1, wherein the carrier comprises a one-piece carrier.

13. The dressing system of claim 12, wherein the one-piece carrier comprises a peripheral border and two pull tabs.

14. The dressing system of claim 1, further comprising:
a catheter securement system, comprising:
a window frame;
a catheter securement; and
a securement carrier.

15. The dressing system of claim 14, wherein the window frame and the catheter securement each have a cut out for a catheter.
